(19) United States
(12) Patent Application Publication
(10) Pub. No.: US 2008/0021506 A1
(43) Pub. Date: Jan. 24, 2008

(54) METHOD AND DEVICE FOR THE ELECTRICAL TREATMENT OF SLEEP APNEA AND SNORING

(75) Inventor: Joseph A. GROCELA, Weston, MA (US)

Correspondence Address: NIXON PEABODY, LLP, 401 9TH STREET, NW, SUITE 900, WASHINGTON, DC, 20004-2128, US

(73) Assignee: MASSACHUSETTS GENERAL HOSPITAL, Charlestown, MA (US)

(21) Appl. No.: 11/746,421

(22) Filed: May 9, 2007

Related U.S. Application Data
(60) Provisional application No. 60/798,749, filed on May 9, 2006.

(51) Int. Cl. A61N 1/36

(52) U.S. Cl. 607/9; 128/200.24; 607/116; 607/148; 607/42

(57) ABSTRACT

A device and method for the treatment of sleep apnea and/or snoring in a human patient includes at least one electrode stimulator for providing direct electrical stimulation to a throat and/or laryngeal muscles of the patient. The stimulator is constructed and arranged to be positioned at the throat and/or laryngeal muscles of the patient, either on the surface or subcutaneously. A power source provides a continuous electrical signal to the stimulator so that the throat and/or laryngeal muscles of the patient are contracted to open the airway of the patient. The power source can be a portable source remote from the electrode or a pacemaker unit also implanted in the patient.

### RELATED APPLICATION

This application claims priority of U.S. Provisional Application No. 60/798,749, entitled “Method and Device For the Electrical Treatment of Sleep Apnea” filed on May 9, 2006.

### BACKGROUND OF THE INVENTION

1. Field of the Invention

The present invention relates to a device for electrically treating sleep apnea and/or snoring, and more particularly, to a portable device and method for continuously contracting and/or stimulating tissue of the tongue and/or laryngeal muscles to unblock the patient's airway during sleep.

2. Description of the Related Art

Sleep apnea affects approximately 1% of women, and 4% of men. According to the National Heart, Lung, and Blood Institute (NHLBI), approximately 18 million people suffer from obstructive and/or non-obstructive sleep apnea. Obstructive sleep apnea, wherein an obstruction, such as the tongue, prevents breathing, affects approximately 12 million people in the United States. Non-obstructive or central sleep apnea, which occurs do to an interruption in the brain signal to breathe, affects approximately 6 million people in the United States. As the population becomes more obese in the United States, these proportions are expected to rise.

In addition to snoring, loss of sleep and productivity are commonplace for those suffering from sleep apnea. In addition, correlation between sleep apnea and automobile accidents due to falling asleep at the wheel has been shown. If untreated, serious complications and long-term effects, such as, hypertension, pulmonary hypertension, right-sided heart failure, and/or stroke can occur.

There are a few general ways to improve the condition: i) weight loss—snoring and sleep apnea are aggravated by excess fatty tissue in the neck and throat area; ii) avoiding alcohol—alcohol tends to relax the throat muscles; iii) use a humidifier—dry air is a throat irritant; iv) stop smoking—smoking irritates the throat and makes breathing more difficult; v) getting adequate sleep—being over-tired exacerbates the problem; and vi) avoid sleeping on the back—people who sleep on their backs tend to snore more because this position narrows the airway. It is better to sleep on one's side if at all possible.

Sleep apnea is currently diagnosed through sleep studies or polysomnography, where waking and apneic cycles are measured overnight. The two primary modes of current treatment for sleep apnea include surgery and a device that provides continuous positive air pressure (CPAP). As treatment, surgery has mixed and lower success rates, and CPAP devices do work, but have substantial drawbacks, e.g. patients feel as if they are suffocating with the mask applied to their nose, loud noise from blowing air, and patient appearance is odd.

The specific therapy for sleep apnea is tailored to the individual patient based on medical history, physical examination, and the results of polysomnography that measures the breathing, heart rate and muscle activity during sleep. Medications are generally not effective in the treatment of sleep apnea. Oxygen administration may safely benefit certain patients, but does not eliminate sleep apnea or prevent daytime sleepiness. Thus, the role of oxygen in the treatment of sleep apnea is controversial, and it is difficult to predict which patients will respond well. It is important that the effectiveness of the selected treatment be verified; this is usually accomplished by polysomnography.

Another treatment option is behavioral therapy. Behavioral changes are an important part of the treatment program, and in mild cases behavioral therapy may be all that is needed. The individual should avoid the use of alcohol, tobacco, and sleeping pills, which make the airway more likely to collapse during sleep and prolong the apneic periods. Overweight persons can benefit from losing weight. Even a 10% weight loss can reduce the number of apneic events for most patients. In some patients with mild sleep apnea (or snoring), breathing pauses occur only when they sleep on their backs. In such cases, using pillows and other devices that help them sleep in a side position is often helpful.

Dental appliances that reposition the lower jaw and the tongue have been helpful to some patients with mild sleep apnea or who snore but do not have apnea. Possible side effects include damage to teeth, soft tissues, and the jaw joint. A dentist or orthodontist is often the one to fit the patient with such a device.

For more serious conditions physical or mechanical therapy is needed. Nasal continuous positive airway pressure (CPAP) is the most common effective treatment for sleep apnea. In this procedure, the patient wears a mask over the nose during sleep, and pressure from an air blower forces air through the nasal passages. The air pressure is adjusted so that it is just enough to prevent the throat from collapsing during sleep. The pressure is constant and continuous. Nasal CPAP prevents airway closure while in use, but apnea episodes return when CPAP is stopped or used improperly.

However, the CPAP devices are loud and cumbersome and have numerous side effects making sleep difficult during use. Variations of the CPAP device have attempt to minimize side effects that sometimes occur, such as throat and nasal irritation and drying, sinus problems, facial skin irritation, abdominal bloating, mask leaks, sore eyes, and headaches. Some versions of CPAP vary the pressure to coincide with the person's breathing pattern, and others start with low pressure, slowly increasing it to allow the person to fall asleep before the full prescribed pressure is applied. Moreover, some of the newer masks are gel-filled to fit better and are less irritating. However, many patients still suffer from claustrophobia because the mouth and/or nose still need to be covered.

Some patients with sleep apnea are treated with surgery. Although several surgical procedures are used to increase the size of the airway, none of them is completely successful or without risks. More than one procedure may need to be tried before the patient realizes any benefits. Some of the more common procedures include removal of adenoids and tonsils (especially in children), nasal polyps or other growths, or other tissue in the airway and correction of structural deformities. Younger patients seem to benefit from these surgical procedures more than older patients.

There are several surgical procedures that can be used in the treatment of sleep apnea, and its companion problem, snoring. Some of these are: somnoplasty, uvulopalatopharyngoplasty (UPPP), laser-assisted uvulopalatoplasty (LAUP), tracheotomy, and tracheostomy. Somnoplasty treats snoring and obstructive sleep apnea by shrinking soft tissues in the upper airway, including the base of tongue, the source of obstruction that is most difficult to treat. This soft tissue often blocks air passages, making breathing difficult. Uvulopalatopharyngoplasty (UPPP) is a surgical procedure that reduces and reshapes the uvula and posterior soft palate in order to enlarge the oropharyngeal airway. This procedure is used to remove excess tissue at the back of the throat (tonsils, uvula, and part of the soft palate). The success of this technique may range from 30% to 50%. The long-term side effects and benefits are not known, and it is difficult to predict which patients will do well with this procedure.

Laser-assisted uvulopalatoplasty (LAUP) is a procedure similar to UPPP, but is an out-patient surgical procedure under local anesthesia involving several sessions spaced several weeks apart. This procedure usually is done to treat snoring but has not been shown to be effective in treating sleep apnea. Less tissue is removed than by the UPPP surgery. Like UPPP, LAUP may decrease or eliminate snoring but not sleep apnea itself. A laser device is used to eliminate tissue in the back of the throat. Elimination of snoring, the primary symptom of sleep apnea, without influencing the condition may carry the risk of delaying the diagnosis and possible treatment of sleep apnea in patients who elect LAUP. To identify possible underlying sleep apnea, sleep studies are usually required before LAUP is performed.

A tracheotomy is a surgical opening made into the trachea for airway management. A tracheostomy is the surgical creation of a stoma from the trachea to the overlying skin. This procedure is used in persons with severe, life- threatening sleep apnea. In this procedure, a small hole is made in the windpipe and a tube is inserted into the opening. This tube stays closed during waking hours and the person breathes and speaks normally. It is opened for sleep so that air flows directly into the lungs, bypassing any upper airway obstruction. Although this procedure is highly effective, it is an extreme measure that is poorly tolerated by patients and rarely used.

In an attempt to provide less invasive treatments numerous implantable devices have been developed. U.S. Pat. No. 6,587,725 discloses a method and apparatus for stimulation of the hypoglossal nerve to treat obstructive sleep apnea. The device uses a plurality of stimulators that are implanted in the patient to form a closed loop nerve stimulator. As with the previous described treatment methods, this treatment is invasive as it must be completely implanted, and it does not use any external devices or percutaneous leads. This is especially invasive for obese patients because due to the larger amount of soft tissue the devices must be implanted deeper so as to be close enough to the nerve to stimulate the same.

A similar problem arises when electrodes are implanted in a patient to stimulate muscle and nerve tissue to help open blocked airways. U.S. Pat. No. 6,345,202 discloses such a device that operates to provide electrical stimulation in a period that corresponds to a natural respiratory rhythm of the patient. The device must be implantable, which is especially invasive for obese patients. Another problem with such a prior art device is that the electrical signals are intermittent and do not provide for a continuous contraction and/or opening of the passage. Moreover, the systems needed to support these devices are complex requiring that the patient be located at a medical facility or clinic during use.

As the cases of sleep apnea arise due to obesity and other factors, there is a need for a long-term sleep apnea treatment that does not require invasive surgery or loud cumbersome devices.

### SUMMARY OF THE INVENTION

One aspect of the present invention is a sleep apnea treatment device that is positionable externally on a patient to stimulate muscle and nerve tissue non-invasively.

Another aspect of the present invention is a sleep apnea treatment device that is portable, allowing for use in home, during travel, etc.

Yet another aspect of the present invention is a sleep apnea treatment device that is silent and non-invasive allowing for minimum discomfort and sleep interruption.

Still yet another aspect of the present invention is to provide a sleep apnea treatment device that allows for continuous muscle contraction to provide a continuously open airway.

Another aspect of the present invention is a sleep apnea treatment device that can be used on a variety of different sized patients. For example, by adjusting the number and position of pads small to obese sized patients can be treated with a single device.

Still another aspect of the present invention is to provide a sleep apnea treatment device that is subcutaneously implantable in the patient.

Yet another aspect of the present invention is to provide a device for preventing snoring that is positionable externally on a patient to stimulate muscle and nerve tissue non-invasively to prevent a user from snoring during sleep.

In accomplishing these and other aspects of the present invention there is provided a device for the treatment of sleep apnea in a human patient. The device includes at least one stimulator for providing direct electrical stimulation to a throat and/or laryngeal muscles of the patient. The stimulator is constructed and arranged to be positioned at the throat and/or laryngeal muscles of the patient. A power source provides a continuous or continuously pulsed electrical signal to the stimulator so that the throat and/or laryngeal muscles of the patient are contracted to open the airway of the patient.

In accomplishing yet another aspect of the present invention there is provided a method for treating sleep apnea in a human patient. The method includes the steps of positioning at least one stimulator for providing direct electrical stimulation at the throat and/or laryngeal muscles of the patient, and providing a continuous electrical signal to the stimulator so that the throat and/or laryngeal muscles of the patient are contracted to open the airway of the patient.

These and other features, aspects, and advantages of the present invention will become more apparent from the following detailed description of the preferred embodiment relative to the accompanied drawings, in which:

### BRIEF DESCRIPTION OF THE DRAWINGS

FIG. 1 is a perspective view of the placement of the conductive pads of the present invention on a human patient.

FIG. 2 illustrates the sleep apnea treatment device of the present invention.

FIGS. 3 and 4 illustrate another embodiment of a sleep apnea treatment device according to the present invention.

### DETAILED DESCRIPTION OF THE PREFERRED EMBODIMENTS

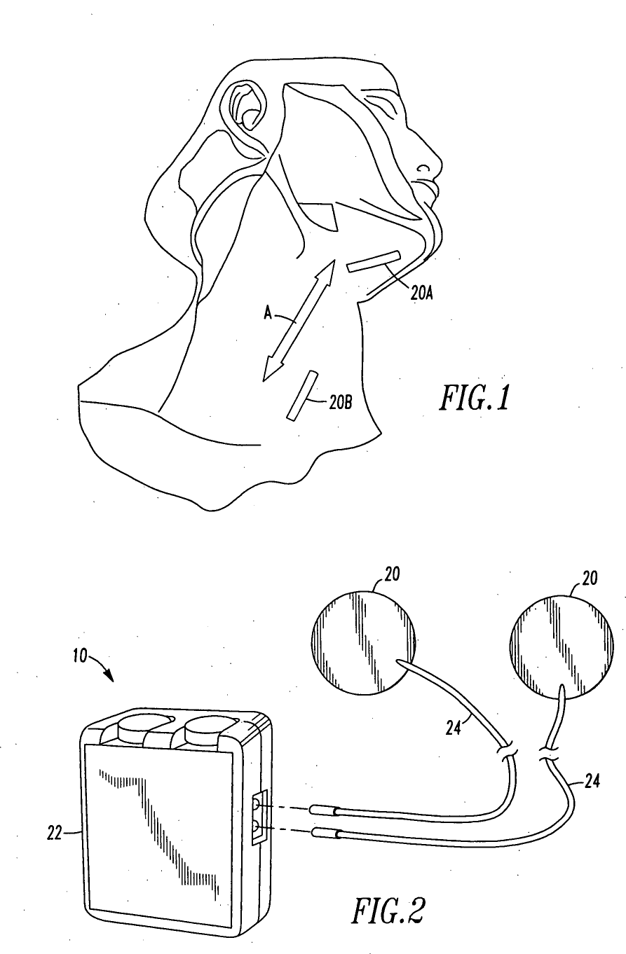

Referring to FIG. 1, in one embodiment of the present invention a portable electronic sleep apnea device includes a plurality of pads 20 disposed above the laryngeal muscles of the patient. When a person lies down, for example, to sleep, the tongue may drop to the back of the throat and the laryngeal muscles may also relax, obstructing the airway. The present invention relies upon electrical stimulation of the throat and laryngeal muscles to provide tonicity and prevent airway collapse or obstruction. Because muscles and nerves conduct electricity relatively well as compared to fat and skin, electricity is passed through the muscles making them contract. This contraction adds muscle tone preventing apnea episodes.

The device can also be used to prevent snoring. When the patient's muscles relax and the airway becomes partially obstructed the patient snores during sleep. By tightening the muscle tone, snoring is decreased significantly and even prevented.

A first pad 20A can be placed beneath the chin. By providing electrical impulses to the pad in the direction of arrow A the tongue muscles will contract tending to make the tongue stick out rather than fall back and obstruct the person's airway. Likewise, by positioning a second pad 20B at the level of the glottis a concentration of electrical impulses will contract the glottis and supporting muscles to prevent obstruction at this level as well. It should be appreciated that additional pads can be placed on the patient depending on the size of the patient and the severity of the apnea episodes.

Referring to FIG. 2, the portable sleep apnea device 10 includes pads 20 and a control unit 22. The device can be portable, driven by batteries located within unit 22. Control unit can be similar to Medtronic Test Simulator Model No. 3625. A plurality of leads 24 communicate with unit 22 at one end and pads 20 at the other end. Depending upon the size of the patient and the severity of the apnea, each lead can have one or more pads attached thereto. Moreover, although only two leads are illustrated additional leads can be provided.

Output jacks, appropriate circuitry, the power source and other components are housed within unit 22. Leads 24 need to be long enough to allow for the unit to be positioned remote from pads 20. For example, during sleep the patient can place the unit on a bedside table or clipped to the waist or pocket of pajamas. The unit could also be positioned on a belt or the electronic circuitry and power source, rather than being located within a separate unit could be disposed directly within the belt, with the same being worn by the patient. As will be described further herein, the unit can also be placed subcutaneously or deeper within the body with an implantable form.

Because the pads are positioned externally on the patients skin a wide range of voltage may be required depending upon the size of the patient and other factors. For example, with an obese patient due to the amount of tissue that may be located at the throat a stronger electronic signal may be required to stimulate the throat and laryngeal muscles. A pediatric patient would require a much gentler impulse. Accordingly, the unit of the present invention should be adjustable to operate within the range of 0 to 100 volts, and/or 10 to 100 mAs. It should be appreciated that the present invention contemplates a wide range of parameters. Dials on the unit allow the patient to adjust the power supply.

Importantly, the electronic impulses should provide a continuous contraction rather than an intermittent pulse. A pulse width within the range of 0-35 pulses per second, and more particularly, between 5-10, has been determined to correctly stimulate the tissue and at the same time not cause discomfort to the patient.

Pads 20 can be disposable and made of a flexible material, such as vinyl. The pads can be a variety of sizes and have numerous different shapes. Moreover, the pads can be cut to fit as long as the connection to lead 24 is not damaged. A gel self-adhesive, or the mechanical equivalent thereof, can be provided on the pad to allow the same to be removably adhered to the patient.

Different applications of the device of the present invention can be used to stimulate the tongue with electrodes or conducting pads placed lateral to each side of the throat or larynx. In addition, the pads can be placed in an anterior/posterior configuration for stimulation as needed. Because the device of the present invention can be operated within a broad range of voltages and pulsing schemes, treatment can be individualized to a patient-specific configuration. Pads 20 can be constructed and arranged to be used as conducting pads for nightly treatment by a patient.

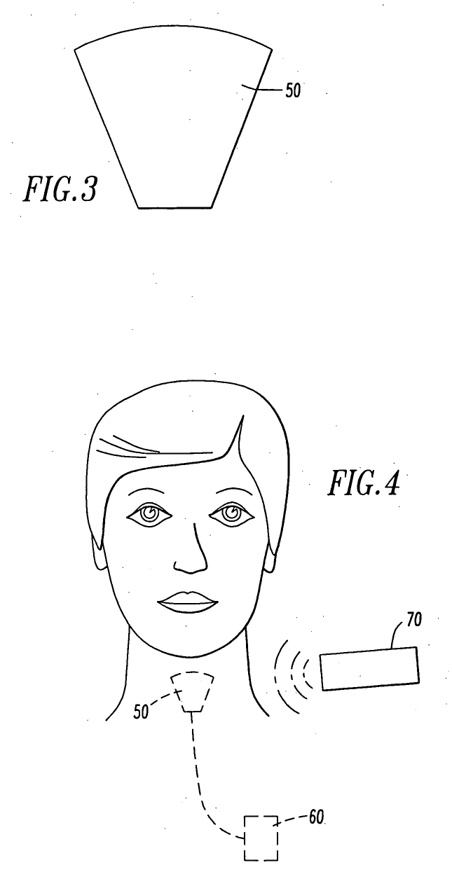

Referring to FIGS. 3 and 4, a subcutaneously implantable electrode device 50 having a pacemaker generator 60 implanted subcutaneously can be used to treat a patient. One or more fan-shaped or flat electrodes 50 can be implanted in the patient in the area above the laryngeal muscles. The parameters of stimulation could then be transcutaneously programmed into a pacemaker type unit 60 individually suited for the patient. In this embodiment, the implantable electrode can be made of a fan-shaped material, such that the maximum area is covered. It should be appreciated that the electrode can take a variety of shapes and sizes and that the wires can be insulated to conduct the impulse. The electrodes can be implanted at a variety of depths depending on the size of the patient. Likewise, more than one implantable electrode can be provided.

Alternatively, implantable electrodes 50 can be stimulated by a remote power source of a portable device 70. As an example, the pulses for stimulation can be made by magnetic induction with an external device and internal magnetic receiver.

Other embodiments of the sleep apnea treatment device are contemplated by the present invention. For example, the conducting pads can be configured to be standalone pads having the circuitry and power source disposed therein. Alternatively, the device could be a collar having the pads placed on the interior thereof such that when the collar is positioned about the neck of the patient the pads are correctly positioned to stimulate the tongue or laryngeal muscles. The device and method of the present invention can also be used in the treatment of other breathing disorders and should not be limited to the treatment of apnea and/or snoring.

Although the present invention has been described in relation to particular embodiments thereof, many other variations and modifications and other uses will become apparent to those skilled in the art. It is preferred therefore, that the present invention be limited not by the specific disclosure herein, but only by the appended claims.

What is claimed is:

1. A device for the treatment of sleep apnea or snoring in a human patient, comprising:
at least one stimulator for direct electrical stimulation of a throat and/or laryngeal muscles of the patient, the at least one electrode being constructed and arranged to be positioned at the throat and/or laryngeal muscles of the patient; and
a power source for providing a continuous electrical signal to the at least one stimulator so that the throat and/or laryngeal muscles of the patient are contracted to open the airway of the patient.

2. The device of claim 1, wherein the at least one stimulator comprises a plurality of conductive electrodes removably positionable above the throat and/or laryngeal muscles of the patient.

3. The device of claim 2, wherein the electrodes are contained in adhesive backed pads.

4. The device of claim 1, wherein the power source is remotely spaced from the at least one stimulator.

5. The device of claim 1, wherein the power source has a pulse width within the range of 0 to 35 pulses per second.

6. The device of claim 1, wherein the at least one stimulator comprises a fan-shaped electrode implantable in the patient at the throat and/or laryngeal muscles of the patient.

7. The device of claim 1, wherein the at least one stimulator comprises a flat electrode implantable in the patient at the throat and/or laryngeal muscles of the patient.

8. The device of claim 5, wherein the power source comprises a pacemaker implanted within the patient.

9. A method for treating sleep apnea or snoring in a human patient, the method comprising the steps of:
positioning at least one stimulator for providing direct electrical stimulation at the throat and/or laryngeal muscles of the patient;
providing a continuous electrical signal to said at least one stimulator so that the throat and/or laryngeal muscles of the patient are contracted to open the airway of the patient.

10. The method of claim 9, wherein the positioning step comprises removably positioning at least one electrode at the throat and/or laryngeal muscles of the patient.

11. The method of claim 10, wherein the at least one electrode is located within an adhesive backed pad and the positioning step comprises attaching the pad to the skin of the patient above the throat and/or laryngeal muscles of the patient.

12. The method of claim 9, wherein the positioning step comprises implanting at least one fan-shaped electrode in the patient above the throat and/or laryngeal muscles of the patient.

13. The method of claim 9, wherein the positioning step comprises implanting at least one flat-shaped electrode in the patient above the throat and/or laryngeal muscles of the patient.

14. The method of claim 12, wherein the step of providing a continuous electrical signal comprises sending an electrical signal from a pacemaker implanted in the patient to the at least one electrode.

15. The method of claim 12, wherein the step of providing a continuous electrical signal comprises sending an electrical signal from a remote power source to the at least one electrode.
